(19) United States
(12) Patent Application Publication
(10) Pub. No.: US 2013/0224077 A1
(43) Pub. Date: Aug. 29, 2013

(54) Distributed Ozone Disinfection System

(71) Applicant: Bruce Edward Hinkle, Newport Beach, CA (US)

(72) Inventor: Bruce Edward Hinkle, Newport Beach, CA (US)

(21) Appl. No.: 13/776,555

(22) Filed: Feb. 25, 2013

Related U.S. Application Data
(60) Provisional application No. 61/603,872, filed on Feb. 27, 2012.
(60) Provisional application No. 61/618,552, filed on Mar. 30, 2012.

(51) Int. Cl. A61L 2/20

(52) U.S. Cl. CPC A61L 2/202
USPC 422/111; 422/119

(57) ABSTRACT

A distributed ozone disinfection system has a central ozone generation system, and ozone and water mixing systems. Each of the ozone and water mixing systems is positionable in a water supply piping at a water supply inlet for a sink faucets or water outlets. The distributed ozone disinfection system has vacuum switches, separate from vacuum switches positionable downstream which are in turn separate from the ozone and water mixing systems, and a plurality of oxidation reduction potential (ORPs) meters. The ORP meters are positionable downstream and separate from the ozone and water mixing systems. Optionally, the ozone and water mixing system includes a vacuum switch coupled with a gas injection venturi device.

This application claims domestic priority from and is a non-provisional of same inventor Bruce Hinkle's U.S. provisional patent application 61/603,872 filed Feb. 27, 2012 entitled Distributed Ozone Disinfection System, the disclosure of which is incorporated herein by reference.

This application claims domestic priority from and is also a non-provisional of same inventor Bruce Hinkle's United States provisional patent application 61/618,552 filed Mar. 30, 2012 entitled Distributed Ozone Disinfection System, the disclosure of which is incorporated herein by reference.

### FIELD OF THE INVENTION

The invention relates generally to ozone disinfection and more particularly to systems and methods facilitating distributed aqueous ozone disinfection for reducing bacteria on materials washed with ozonated water.

### BACKGROUND INFORMATION

Current accepted food preparation practices typically result spreading the bacteria that arrived on a food item to the prepared portions of the food item or to other items. Bacteria can lead to food spoilage and other complications resulting in increased costs.

To combat the spread of bacteria, most establishments rinse food items with water upon arrival and also rinse food preparation surfaces and tools with water throughout the day. Water alone does not eliminate bacteria. However, ozone in water has been demonstrated to be effective at dramatically reducing bacteria on materials washed with that ozonated water. For example, in U.S. Pat. No. 6,458,257, entitled Microorganism Control Of Point Of Use Potable Water Sources to inventor Andrews, issued Oct. 1, 2002, the disclosure of which is incorporated herein by reference, discusses use of an ozone generator for controlling microorganisms.

In a food preparation installation, such as a restaurant or food packaging facility, an ozonated water system typically includes one or more sinks to supply ozonated water from the one or more sinks In installations with several sinks, supplying ozonated water from each sink can be costly. In addition, it may not be physically feasible due to space designed construction and/or aesthetic reasons to provide an ozone generation system at each sink.

Thus, it is desirable to provide a distributed ozone system that would facilitate aqueous ozone disinfection on a distributed basis that is sufficient to be effective at dramatically reducing bacteria on materials and surface areas washed with ozonated water.

### SUMMARY OF THE INVENTION

The embodiments described herein are directed to systems and methods that would facilitate aqueous ozone disinfection on a distributed basis that is sufficient to be effective at dramatically reducing bacteria on materials and surface areas washed with ozonated water. The system provided herein facilitates the distribution of ozonated water to multiple sinks faucets and outlets while keeping the additional hardware and associated costs at each sink at a minimum. In one embodiment, water supplied to a sink faucet is mixed with ozone to produce ozonated water. The system includes a central ozone generation system coupled to a venture system at each sink. The venturi system can be coupled with vacuum flow switch for system activation. The level of ozonation of the water at each sink may be monitored in real-time by an Oxidation-Reduction-Potential (ORP) meter to verify that the ozone level at each sink is sufficient to disinfect any pathogens present. A vacuum switch is positioned in line with the ozone gas tube coupled to the venturi system and coupled to the ozone generation system to prevent production of ozone until after the flow of water has produced vacuum commenced through the venturi injection system.

A distributed ozone disinfection system has a central ozone generation system, and ozone and water mixing systems. Each of the ozone and water mixing systems is configured to mount to and positionable in a water supply piping at a water supply inlet for a sink faucets or water outlets. The distributed ozone disinfection system has vacuum switches, separate vacuum switches positionable downstream which are in turn separate from the ozone and water mixing systems, and a plurality of oxidation reduction potential (ORPs) meters. The ORP meters are positionable downstream of each of the ozone and water mixing systems. Optionally, the ozone and water mixing system includes a vacuum switch coupled with a gas injection venturi device. The ozone generator can be a corona discharge type ozone generator coupled to a special zeolites and desiccant media air filter/dryer device. A control system design can be coupled to the ozone generation system. The control system includes a vacuum switch to activate the transfer relay upon sensing of sufficient ozone gas flow through the vacuum intake of the venturi system which has water flow passing through by water valve opening. This relay switches to activate power to the ozone generation system for ozone gas production to the venturi system. Each of the plurality of ORP meters are electrically coupled to an indicator light at the sink that indicates to the user of the sink that the ORP is high enough to effectively reduce the bacteria in the food or surface areas washed by the ozonated water. Other systems, methods, features and advantages of the example embodiments will be or will become apparent to one with skill in the art upon examination of the following figures and detailed description.

### BRIEF DISCRIPTION OF THE DRAWINGS

The details of the example embodiments, including structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a schematic diagram of a distributed ozone disinfection system.

FIG. 2 is a flow diagram of a distributed ozone disinfection system.

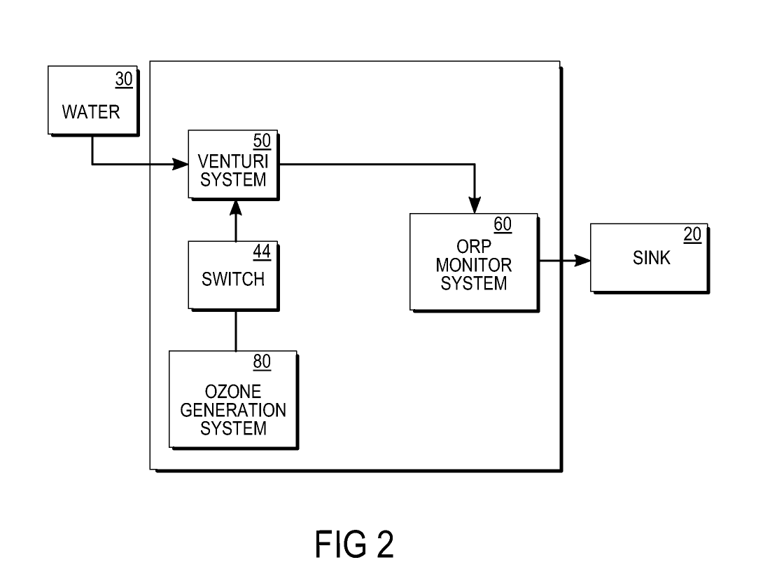

FIG. 3 is a front view of a gas injector venturi device.

FIG. 4 is a schematic of a control diagram of the distributed ozone disinfection system.

FIG. 5 is a plan view of an ozone generator system.

It should be noted that elements of similar structures or functions are generally represented by like reference numerals for illustrative purpose throughout the figures. It should also be noted that the figures are only intended to facilitate the description of the preferred embodiments.

The following callout list of elements can be a useful guide for referencing the callout numbers of the drawings.
System 10
Sinks 20a, 20b And 20n
Basin 22
Faucet 24
On-Off Valve 26
Water Supply Line 30
Water Supply Line 30
Supply Branches 32a, 32b And 32n
Ozonated Water Piping Section 34a, 34b And 34n
Central Ozone Generating System 40
Ozone Supply Tubing 42
Branches 42a, 42b And 42n
Vacuum Switch 44
Line 45
Venturi System 50
Injector Inlet 52
Injection Chamber 53
Injector Outlet 54
Gas Injection Port 56
Orp Monitoring System 60
Orp Sensor 62
Line 63
Orp Meter 64
Orp Indicator Light 66
Control Board Ozone Generation Circuit 70
Transfer Relay 71
Indicator Light 72
Power Switch 73
Power Indicator Light 74
Ac-To-Dc Power Transformer 75
Ozone Generation System 80
Ozone Generator 82
Air Pump 84
De-Humidifier 86
Fan 88

### DETAILED DESCRIPTION OF THE PREFERRED EMBODIMENT

Each of the additional features and teaching disclosed below can be utilized separately or in conjunction with other features and teachings to produce systems and methods to facilitate distributed aqueous ozone disinfection. Representative examples of the present invention, which utilized many of these additional features and teachings both separately and in combination, will now be described in further detail with reference to the attached drawings. This detailed description is merely intended to teach a person of skill in aqueous ozone disinfection, the art further details practicing preferred aspects of the present teachings and is not intended to limit the scope of the invention. Therefore, combinations of features and steps disclosed in the following detail description may not be necessary to practice the invention in the broadest sense, and are instead taught merely to particularly describe representative examples of the present teachings.

Moreover, the various features of the representative examples and the dependent claims may be combined in ways that are not specifically and explicitly enumerated in order to provide additional useful embodiments of the present teachings. In addition, it is expressly noted that all features disclosed in the description and/or the claims are intended to be disclosed separately and independently from each other for purpose of original disclosure, as well as for the purpose of restricting the claimed subject matter independent of the compositions of the features in the embodiments and/or the claims. It is also expressly noted that all value ranges or indications of groups of entitles disclose every possible intermediate value or intermediate entity for the purpose of original disclosure, as well as for the purpose of restricting the claimed subject matter.

The systems and methods provided herein address distribution of ozonated water aqueous ozone to multiple sinks and outlets while keeping the additional hardware and associated costs for installation at a minimum. The present systems and methods also allow the user to verify that the ozone level at each sink is sufficient to disinfect any pathogens present. The present system is particularly suited to use in restaurants, grocery markets, commissaries, hospitals, nursing homes, etc. for ensuring microbial disinfection needed and the freshness and safety of the food.

To keep the added hardware at each sink or water outlet at a minimum, the ease of installation and the price per unit cost low, the ozone is generated at a central location in the facility and piped through tubes to a plurality of sinks or outlets. In a preferred embodiment, up to ten sinks faucets 24 or outlets are preferably supplied with ozone from a single ozone generation system at a distance of preferably up to 175 feet from the location where the ozone is generated. Just before the cold water pipe enters the sink, the water pipe and the ozone tube are connected via a venturi system junction box that mixes the ozone into the water. At the output of the venturi system is an optional oxidation reduction potential (ORP) sensor that measures the level of ozone in the water included is a digital read out of ORP level when the ozone level is appropriate for pathogen reduction, the sensor will cause a light to go on near the sink so that a user can be sure that sufficient aqueous ozone is being generated.

In order that ozone is generated only when it is needed, an air vacuum flow switch attached to the ozone gas feed tube is coupled to the venturi system installed near the sink. This is used to determined when sufficient cold water is flowing into the sink through the faucet. A wire connects this switch to the central ozone generating device. When the vacuum switch coupled to venturi device detects water flowing into the sink creating vacuum, it signals the ozone generating device to begin generating ozone. A small amount of water will flow into the sink before the ozone reaches the sink. This will vary depending on the flow rate of the water and pressure.

To produce the ozone gas the centralized device has a pump that pushes ambient air into the device, a dehumidifier to ensure that the dew point of the pumped in air is sufficiently low for the ozone generator to work effectively, and an ozone generator that uses the corona discharge method for producing ozone. A fan 88 is included to ensure that the device does not overheat.

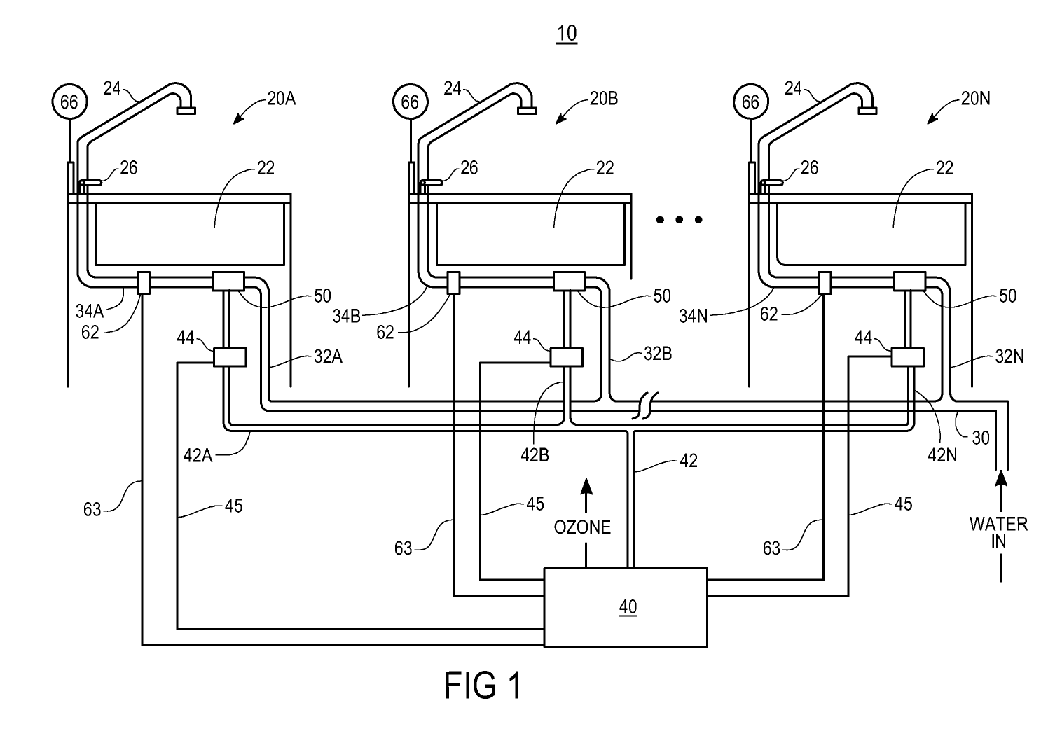

A system 10, as shown in FIG. 1, for disinfection of pathogens on food items and other materials and tools encountered during food preparation and packaging in restaurants and markets, and the like, is comprised of a plurality of N sinks, namely sink 20A, sink 20B and sink 20N coupled to water supply line 30. Each sink includes a basin 22, a faucet, and an on-off valve 26. The water supply line 30 branches off to each sink 20A, 20B and 20N at supply branches 32A, 32B and 32N. Each supply branch includes a venturi system 50 where the water is mixed with ozone from a central ozone generating system 40 to produce ozonated water. The venturi systems 50 are coupled to the central ozone generating system 40 via ozone supply tubing 42 which branches off at branches 42A, 42B and 42N to supply ozone to each venturi system 50. A vacuum switch 44 is coupled to the venturi system 50 and coupled to the central ozone generating system 40 electrically along line 45 or wirelessly. Alternatively, a water flow switch may instead be positioned upstream of the supply branches 32A, 32B and 32N in the water supply line 30 to detect flow through any of the supply branches 32A, 32B and 32N to activate the system. An ORP sensor 62 of an ORP monitoring system 60 is also positioned downstream of the venturi system 50 and may be coupled to the central ozone generating system 40 electrically along line 63 or wirelessly.

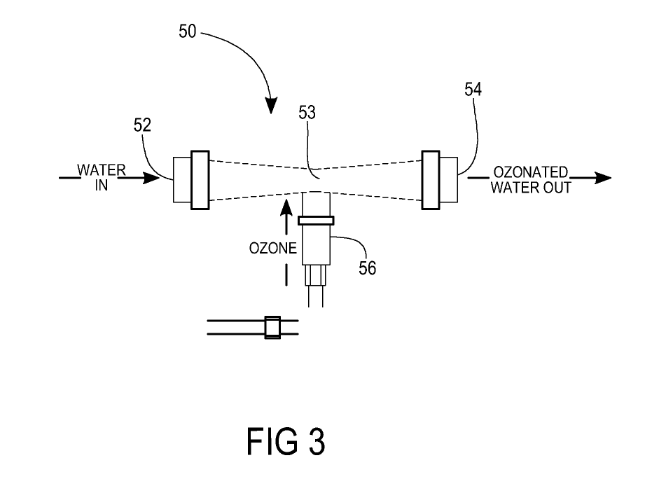

The venturi system 50 is preferably a gas injector venturi device, such as a Mazzei® venturi-type, differential pressure injector. See, e.g., U.S. Pat. No. 5,863,128. As depicted in greater detail in FIG. 3, the gas injector venturi device 50 narrows as it transitions from an injector inlet 52 to an injection chamber and them widens as the injector 50 transitions from the injection chamber 53 to an injector outlet 54. Located at the injection chamber 53 is a gas injection port 56. An ozone gas supply line 42 (FIG. 1), which extends to each sink, is coupled to the vacuum switch 44 which is coupled to the injection port 56. Pressurized water entering the inlet 52 of the injector 50 changes to a high velocity jet stream as it passes through the injection chamber 53 drawing the ozone gas in through the injection port 56 via air vacuum to be entrained or dissolved in the pressurized water.

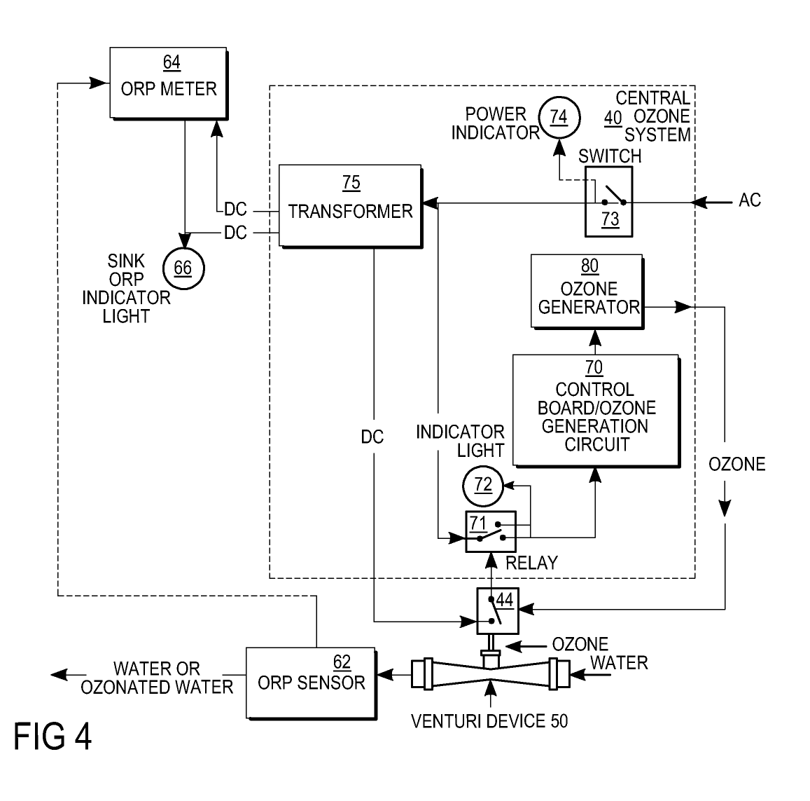

Referring to FIGS. 1 and 4, the ozonated water exits the venturi device 50 and continues through an ozonated water piping section 34A, 34B and 34N at each sink faucet/outlet. An Oxidation-Reduction-Potential (ORP) monitor system 60 includes a sensor 62 positioned downstream of the venturi device 50. The ORP sensor 62 includes a probe extending into the flow of ozonated water in the ozonated water piping section 34A, ozonated water piping section 34B and ozonated water piping section 34N. An ORP meter 64 analyzes the data from ORP sensor 62 to determine if the level of aqueous ozone in the water is at level sufficient for pathogen reduction. At proper levels, the ORP meter 64 will cause an ORP indicator light 66 to turn on at or near the sink so as to indicate to the user that sufficient aqueous ozone is being generated.

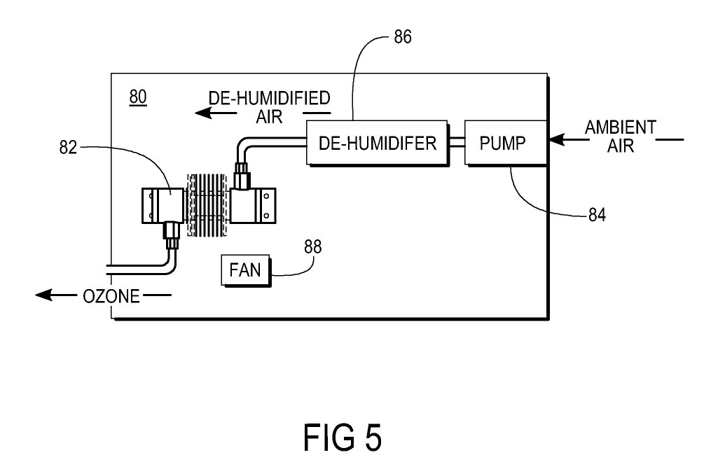

As depicted in FIGS. 4 and 5, the central ozone generating system 40 includes an ozone generation system 80 that includes an ozone generator 82 having an inlet and outlet. The ozone generator 82 is preferably a corona discharge type ozone generator. The ozone generator 82 is coupled at its inlet to an air pump 84 and a de-humidifier 86. The central ozone generating system 40 includes a control board ozone generation circuit 70 coupled to the ozone generation system 80 and configured to control the operation of the generation system 80.

The control logic for the central ozone generating system 40 is depicted in FIG. 4. As depicted, AC power at 120V, 60 Hz is supplied to the system 40 and a user controlled mechanical switch 73 for interrupting power to the system 40 is provided. Included in the switch 73 is a power indicator light 74. An AC-to-DC power transformer 75 is coupled to the switch 73 and provides 24V DC power to the ORP (oxidation reduction potential) meter 64, an ORP indicator light 66, and a transfer relay 71. As depicted, when the vacuum switch 44 detects ozone gas flowing, it closes the DC circuit that in turn closes (activates) the transfer relay switch 71, giving AC power to the control board ozone generation circuit 70 and the ozone generator 80. The control board ozone generation circuit 70 and ozone generation system 80 are coupled in series with the transfer relay 71 to generate ozone once the relay 71 is activated to power the control board ozone generation circuit 70. An indicator light 72 connected to the relay 71 indicates whether ozone is being generated (green), if not then (red), based on whether the transfer relay is open (red) or closed (green).

In operation, pressurized water is supplied to the system so that water is flowing through the venturi system. When the power switch 73 is turned on for the control board ozone generation circuit 70, the vacuum switch 44 senses whether ozone gas is being fed into and flowing through the venturi system 50 for ozone to be mixed with water supplied to the faucet or to the outlet. If vacuum is detected by the vacuum switch 44, the vacuum switch activates the transfer relay 71. As a result, the transfer relay 71 closes and powers the control board ozone generation circuit 70 which causes the ozone generator 82 to generate ozone gas. With the transfer relay 71 activated, the indicator light 72 is illuminated green. If the vacuum switch 44 does not detect vacuum flow through the gas piping system, the vacuum switch 44 will not activate the transfer relay 71 and, thus, the ozone generation circuit 70 will not be powered to cause the ozone generator 82 to generate ozone. With the transfer relay 71 remaining open, the indicator light 72 is illuminated red. Vacuum switch 44 is a pressure switch of modest specification and does not require extreme pressures such as a perfect vacuum. A vacuum here can be any a difference in pressure and can be slight. The ozone generator 82 can generate ozone gas that can be stored in a reservoir before traveling to the vacuum switch 44.

The example embodiments provided herein, however, are merely intended as illustrative examples and not to be limiting in any way. Moreover, one skilled in the art of aqueous ozone disinfection will readily recognize that familiar systems can be equally adapted with appropriate modification of parameters.

In the foregoing specification, the invention has been described with reference to specific embodiments thereof. It will, however, be evident that various modifications and changes may be thereto without departing from the broader spirit and scope of the invention. For example, the reader is to understand that the specific ordering and combination of process actions shown in the process flow diagrams described herein is merely illustrative, unless otherwise stated, and the invention can be performed using different or additional process actions or a different combination or ordering of process actions. As another example, each features of one embodiment can be mixed and matched with other features shown in other embodiments.

Features and processes known to those of ordinary skill may similarly be incorporated as desired. Additionally and obviously, features may be added or subtracted as desired. Accordingly, the invention is not to be restricted except in light of the attached claims and their equivalents.

1. A distributed ozone disinfection system comprising:
a. a central ozone generation system;
b. a plurality of ozone and water mixing systems, each of the plurality of ozone and water mixing systems configured to mount to a water supply piping at a water supply inlet for a sink faucets or water outlets;
c. a plurality of vacuum switches, each of the plurality of vacuum switches positionable downstream of each of the plurality of ozone and water mixing systems, and a plurality of oxidation reduction potential (ORPs) meters, wherein each of the plurality of ORPs are positionable downstream of each of the plurality ozone and water mixing systems.

2. The system of claim 1, wherein the ozone and water mixing system includes a vacuum switch coupled with a gas injection venturi device.

3. The system of claim 1, wherein the ozone generation system includes an ozone generator, wherein the ozone generator is a corona discharge type ozone generator coupled to a special zeolites and desiccant media air filter/dryer device.

4. The system of claim 1, further comprising a control system design coupled to the ozone generation system.

5. The system of claim 4, wherein the control system includes a vacuum switch to activate the transfer relay upon sensing of sufficient ozone gas flow through the vacuum intake of the venturi system which has water flow passing through by water valve opening, wherein the relay switches to activate power to the ozone generation system for ozone gas production introduction to the venturi system.

6. The system of claim 5, wherein each of the plurality of ORP meters are electrically coupled to an indicator light at the sink that indicates to the user of the sink that the ORP is high enough to effectively reduce the bacteria in the food or surface areas washed by the ozonated water.
